(19) United States
(12) Patent Application Publication
(10) Pub. No.: US 2011/0251143 A1
(43) Pub. Date: Oct. 13, 2011

(54) STABLE ELSAMITRUCIN SALT FORMULATIONS

(75) Inventors: Ashok Gore, Tustin, CA (US); Kwok Yin Tsang, Irvine, CA (US)

(73) Assignee: Spectrum Pharmaceuticals, Inc., Irvine, CA (US)

(21) Appl. No.: 12/809,575

(22) PCT Filed: Dec. 19, 2008
(86) PCT No.: PCT/US2008/087683

§ 371 Date: Jun. 23, 2011

Related U.S. Application Data
(60) Provisional application No. 61/015,183, filed on Dec. 19, 2007.

(51) Int. Cl. A61K 31/7048; A61P 35/00

(52) U.S. Cl. 514/27

(57) ABSTRACT

Formulations containing stable forms of elsamitrucin salts are provided. These formulations are useful for treating neoplastic diseases and conditions.

### CROSS-REFERENCE TO RELATED APPLICATIONS

This application claims priority to U.S. Provisional Patent Application No. 61/015,183 filed Dec. 19, 2007. The contents of this application is incorporated by reference herein in its entirety.

### FIELD OF THE INVENTION

The present disclosure relates to elsamitrucin formulations useful for parenteral administration to treat neoplastic diseases and conditions.

### BACKGROUND OF THE INVENTION

Elsamitrucin is a heterocyclic antineoplastic antibiotic isolated from the gram positive bacterium Actinomycete strain J907-21 as described in U.S. Pat. Nos. 4,518,589 and 4,572,895 which are incorporated herein by reference for all they disclose related to the natural history, chemical composition, methods of preparing and bioactivity of elsamitrucin. Elsamitrucin intercalates into DNA at guanine-cytosine (G-C)-rich sequences and inhibits topoisomerase I and II, resulting in single-strand breaks and inhibition of DNA replication. Elsamitrucin possesses significant oncolytic activity against metastatic cancer of the breast, colon and rectum, non-small cell lung and ovary and in patients with relapsed or refractory non-Hodgkin's lymphoma.

Elsamitrucin is known chemically as benzo(h)(1)benzopyrano(5,4,3-cde)(1)ebnzopyran-5,12-dione,10((2-O-(2-amino-2,6-dideoxy-3-O-methyl-alpha-D-galactopyranosyl)-6-deoxy-3-C-methyl-beta-D-galactopyranosyl)oxy)-6-hydroxy-1-methyl, and has the structure generally depicted in Formula I. Elsamitrucin is also known as 10-O-elsaminosylelsarosylchartarin, BBM 2478A, BMY-28090, SPI-28090, BRN 5214813, elsamicin A, elsamitrucina, and elsamitrucine.

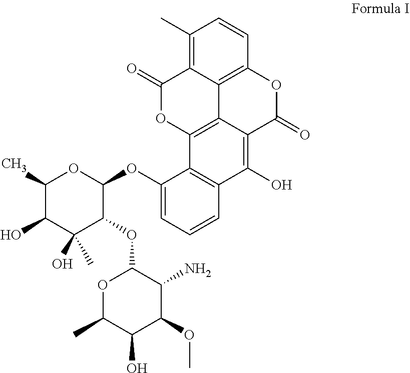

The prior art lyophilized elsamitrucin powder is provided with succinic acid with addition of sterile water. This forms an elsamitrucin salt in situ by dissolving elsamitrucin base in an organic solvent and then adding sufficient aqueous succinic acid to form a 1:1 solution of solubilized free base to acid. The resulting elsamitrucin-succinic acid solution is then adjusted to a pH of between 3.5 and 4.5 and mixed with a bulking agent such as mannitol to enhance stability prior to lyophilization (see for example U.S. Pat. No. 5,508,268). Stable elsamitrucin salts in powder form (crystalline or amorphous) are not presently available thus all highly soluble elsamitrucin pharmaceutical compositions must be prepared in situ using the free base. Elsamitrucin is typically administered parenterally (generally intravenously) to animals, including humans and is supplied as a lyophilized powder that is reconstituted with sterile water for injection immediately prior to use due to the prior art in situ formed salts' inherent instability.

Therefore, there is a need for formulations which contain stable elsamitrucin salts. These formulations should contain salts able to be prepared without the use of the free base and the corresponding organic solvents required to solubilize the free base in situ.

### SUMMARY OF THE INVENTION

The present disclosure relates to formulations containing water soluble, solid elsamitrucin salts which are useful for parenteral administration to treat neoplastic diseases and conditions.

In one embodiment of the present disclosure, the formulation comprises a solution of at least one stable solid elsamitrucin salt and a pharmaceutically acceptable carrier.

In another embodiment of the present disclosure, the formulation does not require a buffer to maintain a solution pH.

In another embodiment of the present disclosure, the formulation does not require a stabilizing antioxidant.

In another embodiment of the present disclosure, the formulation further comprises an osmotic pressure adjusting agent.

In another embodiment of the present disclosure, the formulation further comprises an agent to set the pH between about 3.5 to about 4.5.

In another embodiment of the present disclosure, the formulation's pH is about 4.0.

In another embodiment of the present disclosure, the formulation's solid elsamitrucin salt is selected from the group consisting of elsamitrucin lactate, elsamitrucin fumarate, elsamitrucin maleate, elsamitrucin succinate, elsamitrucin tartrate, elsamitrucin tosylate, elsamitrucin methanesulfonate, elsamitrucin benzoate, elsamitrucin salicylate, elsamitrucin hydrochloride, elsamitrucin sulfate, and elsamitrucin phosphate.

In another embodiment of the present disclosure, the formulation's solid elsamitrucin salt is elsamitrucin tosylate.

In another embodiment of the present disclosure, the pharmaceutically acceptable carrier is water or saline.

These and other objects, advantages and features of the disclosure will be more fully understood and appreciated by reference to the written specification.

### BRIEF DESCRIPTION OF THE DRAWINGS

FIG. 1: Depicts Elsamitrucin Tosylate re-crystallized from 1:1 mixture of acetonitrile:water made in accordance with the teachings of the present disclosure.

FIGS. 2A and 2B show the potency of 2.5 mL Elsamitrucin F2 RTU Dosage Form at 5° C. versus time.

FIGS. 3A and 3B show the potency of 2.5 mL Elsamitrucin F2 RTU Dosage Form at 25° C. versus time.

FIGS. 4A and 4B show the potency of 2.5 mL Elsamitrucin F2 RTU Dosage Form at 40° C. versus time.

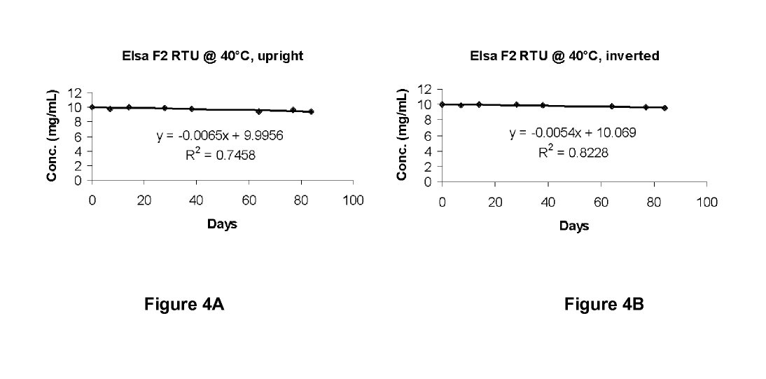

FIGS. 5A and 5B show the potency of 2.5 mL Elsamitrucin F2 RTU Dosage Form at 60° C. versus time.

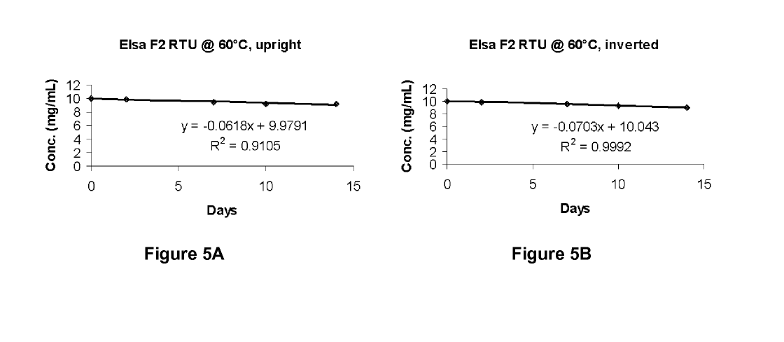

FIG. 6 shows the Arrhenius Plot of Elsamitrucin F2 RTU Dosage Form in Inverted Position.

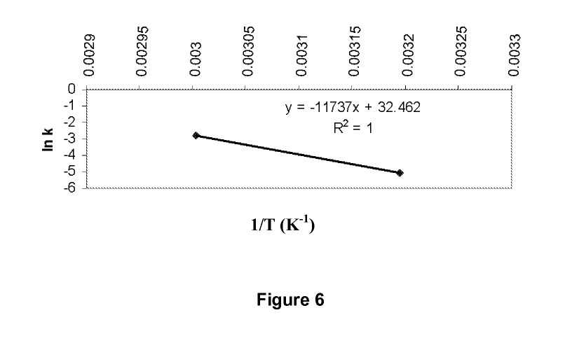

FIG. 7 shows the Arrhenius Plot of Elsamitrucin F2 RTU Dosage Form in Upright Position.

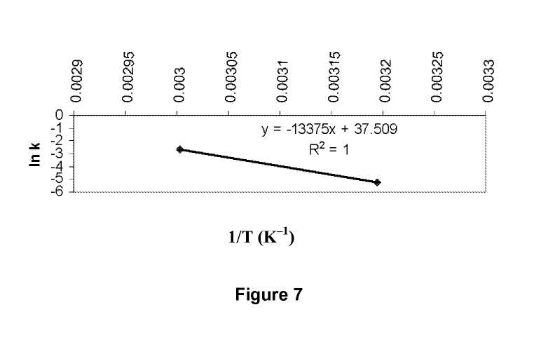

### DEFINITION OF TERMS

Prior to setting forth the disclosure, it may be helpful to provide an understanding of certain terms that will be used hereinafter.

Analog(s): As used herein “analog(s)” include compounds having structural similarity to another compound. For example, the anti-viral compound acyclovir is a nucleoside analog and is structurally similar to the nucleoside guanosine which is derived from the base guanine. Thus acyclovir mimics guanosine (is “analogous with” biologically) and interferes with DNA synthesis by replacing (competing with) guanosine residues in the viral nucleic acid and prevents translation/transcription. Thus compounds having structural similarity to another (a parent compound) that mimic the biological or chemical activity of the parent compound are analogs. There are no minimum or maximum numbers of elemental or functional group substitutions required to qualify as an analog as used herein providing the analog is capable of mimicking, in some relevant fashion, either identically, complementary or competitively, with the biological or chemical properties of the parent compound. Analogs can be, and often are, derivatives of the parent compound (see “derivative” infra). Analogs of the compounds disclosed herein may have equal, less or greater activity than their parent compounds.

Derivative: As used herein a “derivative” is a compound made from (derived from), either naturally or synthetically, a parent compound. A derivative may be an analog (see “analog” supra) and thus may possess similar chemical or biological activity. However, as used herein, a derivative does not necessarily have to mimic the activity of the parent compound. There are no minimum or maximum numbers of elemental or functional group substitutions required to qualify as a derivative. As an example, the antiviral compound ganclovir is a derivative of acyclovir. Ganclovir has a different spectrum of anti-viral activity from that of acyclovir as well as different toxicological properties. Derivatives of the compounds disclosed herein may have equal, less, greater or no similar activity to their parent compounds.

Elsamitrucin: As used herein, the term “elsamitrucin” refers to an anti-neoplastic composition having a molecular weight of approximately 825.83 Da and is known chemically as benzo(h)(1)benzopyrano(5,4,3-cde)(1)ebnzopyran-5,12-dione,10((2-O-(2-amino-2,6-dideoxy-3-O-methyl-alpha-D-galactopyranosyl)-6-deoxy-3-C-methyl-beta-D-galactopyranosyl)oxy)-6-hydroxy-1-methyl, and has the structure generally depicted in Formula I. Elsamitrucin is also known as 10-O-elsaminosylelsarosylchartarin, BBM 2478A, BMY-28090, SPI-28090, BRN 5214813, elsamicin A, elsamitrucina, and elsamitrucine. See U.S. Pat. Nos. 4,518,589 and 4,572,895 for methods of isolating and characterizing elsamitrucin from natural sources. See also Konishi M, Sugawara K, Kofu F, Nishiyama Y, Tomita K, Miyaki T, Kawaguchi H.1986. Elsamicins, new antitumor antibiotics related to chartreusin I. Production, isolation, characterization and antitumor activity. J. Antibiot. (Tokyo) June; 39(6):784-91.

Formulation: As used herein the term formulation refers to a pharmaceutically acceptable preparation comprising one or more of the elsamitrucin salts of the present disclosure and at least one pharmaceutically acceptable carrier such as, but not limited to water for injection or saline. Moreover, the formulations of the present disclosure may also include stabilizers, preservatives, or additional therapeutic agents. The pharmaceutical formulations of the present disclosure may be administered by any means known to those skilled in the art and are ideally suited for intravenous administration or infection into the skin, muscle or other tissues of the body. The pharmaceutical formulation may be intended for oral administration.

Salt: As used herein a “salt” or “salts” include any compounds that result from replacement of part or all of the acid hydrogen of an acid by a metal or a group acting like a metal: an ionic crystalline compound. In this case, the salt is a product of a free base and an organic acid that can exist as a stable solid and does not include pseudo salts, or salts made in situ, which only exist in the solution.

Suitable salt form(s): As used herein, the term “suitable salt form(s)” means an elsamitrucin salt prepared in stable solid state either as amorphous or crystalline form.

Solid or solid salt: As used herein the term solid or solid salt refers to an elsamitrucin salt existing in a solid state and having less than 30% residual moisture, preferably less than 10% residual moisture and more preferably less than 5% residual moisture. As used herein “moisture” refers to water or an organic solvent. The term “solid” is also used herein to differentiate the elsamitrucin salts of the present disclosure from salts formed in situ and exist primarily in the aqueous phase. Further, the present solid salts are not the product of freeze drying or lyophilization.

Stable: As used herein “stable” refers to an elsamitrucin salt or a parenteral elsamitrucin salt-containing formulation (made by method other than in situ salt formation) wherein the elsamitrucin salt retains NMR data showing a near perfect 1:1 salt ratio (thus indicating no decomposition in the solid state) during drying at elevated temperatures at 75° C. for nine hours or more preferably 98° C. overnight. Moreover, stable as used herein refers to elsamitrucin salt-contained in a parenteral formulation that retains at least 90% of its anti-neoplastic activity as determined by in vitro growth inhibition testing (see Example 4) for at least 24 months in the solid form and for 18 months in the liquid form at a suitable storage temperature.

### DETAILED DESCRIPTION OF THE INVENTION

Elsamitrucin and structurally related antibiotics bind to GC-rich tracts in DNA, with a clear preference for B-DNA over Z-DNA. They inhibit RNA synthesis and cause single-strand scission of DNA via the formation of free radicals. Elsamitrucin can also be regarded as the most potent inhibitor of topoisomerase II reported so far and can inhibit the formation of several DNA-protein complexes. Elsamitrucin binds to the P1 and P2 promoter regions of the c-myc oncogene inhibits the binding of the Sp1 transcription factor, thus inhibiting transcription.

Elsamitrucin has shown activity in patients with relapsed or refractory non-Hodgkin's lymphoma and in vivo activity against a wide range of murine neoplasmas including leukemia P388, leukemia L1210, and melanoma B16 and M5076, as well as against MX1 and HCT116 xenografts (see for example Raber M N, Newman R A, Newman B M, Gayer R C, Schacter LP1992 Phase I trial and clinical pharmacology of elsamitrucin. Cancer Res. March 15; 52(6):1406-10).

Additionally, experimental treatment of refractory/relapsed non-Hodgkin's lymphoma has demonstrated that elsamtrucin-associated toxicity is relatively mild and consisted mainly of asthenia, nausea and vomiting and did not include myelosuppression. The activity of elsamitrucin and its lack of myelosuppression suggest utility in this disease especially when combined with other proven agents. (see Allen S L, Schacter L P, Lichtman S M, Bukowski R, Fusco D, Hensley M, O'Dwyer P, Mittelman A, Rosenbloom B, Huybensz S. 1996. Phase II study of elsamitrucin (BMY-28090) for the treatment of patients with refractory/relapsed non-Hodgkin's lymphoma. Invest. New Drugs. 14(2):213-7.

The in vitro activity of elsamitrucin was also investigated as compared with that of doxorubicin (DX) on two sensitive breast cancer cell lines: one estrogen receptor-positive (ER+, MCF7) and one estrogen receptor-negative (ER−, MDA-MB-231) line, and on a DX-resistant subline (MCF7DX). The activity of the two drugs was also investigated on 19 clinical breast cancer specimens from untreated patients. The drugs were tested at pharamcologically relevant concentrations, as calculated from the area under the curve for a 3 h exposure to the lethal dose producing 10% mortality (LD10) in mice, and at 10- and 100-fold concentrations. In DX-sensitive lines, a greater inhibition of RNA and DNA precursor incorporation, as well as of cell proliferation, was caused by elsamitrucin than by DX. Moreover, the antiproliferative effect was 10-fold higher in the ER+ MCF7 than in the ER− MDA-MB-231 cell line (IC50: 0.25 versus 0.21 micrograms/ml). Elsamitrucin was cross-resistant to DX in the MCF7DX subline. In clinical specimens, effects on DNA precursor incorporation were more often observed for elsamitrucin than for DX at the same drug concentrations. The in vitro sensitivity to elsamitrucin was more pronounced for ER+than for ER− tumors: minimal inhibiting concentrations of the drug were 0.1 and 3.5 micrograms/ml, respectively, in the two groups. These in vitro results would indicate a promising role for elsamitrucin in clinical treatment, mainly of ER+ breast cancer patients (see Silvestrini R, Sanfilippo O, Zaffaroni N, De Marco C, Catania S. 1992. Activity of a chartreusin analog, elsamitrucin, on breast cancer cells. Anticancer Drugs. December; 3(6):677-81).

U.S. Pat. No. 5,508,268 issued Apr. 16, 1996 to Nassar et al. assigned to Bristol-Myers Squibb (hereinafter the '268 patent) discloses parenteral formulations comprising elsamitrucin base, an organic acid, a stabilizer and a buffer. The elsamitrucin compositions disclosed therein were prepared using various organic acids including hydrochloric, L(+)-lactic, L-tartaric, D-glucuronic, methane-sulfonic, adipic and succinic with the succinic acid being preferred. The elsamitrucin compositions are prepared according to the teachings of an example occurring at column 4 lines 5-30. In this example, only the succinate salt is described. Specifically, the '268 patent, in accordance with the disclosure therein, the elsamitrucin salt is formed in situ using an organic acid in combination with at least one reducing agent (preservative) and the pH adjusted to approximately 4. The resulting solution was filtered and retained in the liquid state for stability testing. In other embodiments disclosed in the '268 patent the organic acid, elsamitrucin base, reducing agent and other suitable pharmaceutical excipients such as, but not limited to sugars, are admixed in solution and the resulting composition is lyophilized.

However, the '268 patent does not disclose, discuss or teach stable solid elsamitrucin salts. In sharp contrast to the teachings of the '268 patent the present inventors have discovered methods that provide stable solid elsamitrucin salts made using elsamitrucin base and selected organic acids. The resulting compositions made in accordance with the teachings of the present disclosure are solid, dry or partially dried elsamitrucin salt powders, as opposed to lyophilizates described in the '268 patent. Thus, the elsamitrucin salt compositions of the present disclosure are true salts in solid state, not in situ solutions containing a solubilized base and organic acid admixture.

The present disclosure offers numerous advantages over in situ formed admixtures as described in the '268 patent. First, the elsamitrucin salts made in accordance with the teachings of the present disclosure can be carefully analyzed for impurities and refined as needed to meet exceedingly high governmental regulations. Moreover, the true salts of the present disclosure can be precisely weighed and dissolved in suitable pharmaceutical carriers such as Water for Injection. The selected salts themselves are extremely stable when stored in the solid state and have extended shelf lives as do their corresponding solubilized solutions. Thus, parenteral solutions can be prepared using the elsamitrucin salts of the present disclosure and stored for extended periods of time.

In one embodiment of the present disclosure the formulation comprises at least one stable solid elsamitrucin salt and a pharmaceutically acceptable carrier.

In another embodiment of the present disclosure the formulation does not require a buffer to maintain a solution pH.

In another embodiment of the present disclosure the formulation does not require a stabilizing antioxidant.

In another embodiment of the present disclosure, the formulation further comprises an osmotic pressure adjusting agent.

In another embodiment of the present disclosure, the formulation further comprises an agent to set the pH between about 3.5 to about 4.5.

In another embodiment of the present disclosure, the formulation's pH is about 4.0.

In another embodiment of the present disclosure, the formulation's solid elsamitrucin salt is selected from the group consisting of elsamitrucin lactate, elsamitrucin fumarate, elsamitrucin maleate, elsamitrucin succinate, elsamitrucin tartrate, elsamitrucin tosylate, elsamitrucin methanesulfonate, elsamitrucin benzoate, elsamitrucin salicylate, elsamitrucin hydrochloride, elsamitrucin sulfate, and elsamitrucin phosphate.

In another embodiment of the present disclosure, the formulation's solid elsamitrucin salt is elsamitrucin tosylate.

In another embodiment of the present disclosure, the pharmaceutically acceptable carrier is water or saline.

The presently disclosed formulations may not require a buffer to maintain the pH. Buffering agents are usually either the weak acid or weak base that would comprise a buffer solution. Buffering agents are usually added to water to form buffer solutions. They are the substances that are responsible for the buffering seen in these solutions. These agents are added to substances that are to be placed into acidic or basic conditions in order to stabilize the substance. For example, buffered aspirin has a buffering agent, such as MgO, that will maintain the pH of the aspirin as it passes through the stomach of the patient. Another use of a buffering agent is in antacid tablets, whose primary purpose is to lower the acidity of the stomach. Examples of buffering agents are but not limited to potassium dihydrogen phosphate, succinic acid, L(+)-lactic acid, and L-tartaric acid.

When making the present formulations one simply may use an agent to set but not necessarily maintain the desired pH. Acids and bases can be used for this purpose. An example of one such agent is a strong base such as NaOH. A strong base is a basic chemical compound that is able to deprotonate very weak acids in an acid-base reaction. Compounds with a pKₐ of more than about 13 are called strong bases. Common examples of strong bases are the hydroxides of alkali metals and alkaline earth metals like NaOH and Ca(OH)₂.

One of ordinary skill in the art may determine the desired pH or pH range(s) for the elsamitrucin formulation and set this pH or pH range(s) with a pH adjusting agent if necessary. For the purposes of the present disclosure, the pH ranges can be, but not limited to, about 3.5 to about 4.5 and about 2.0 to about 4.0. In another embodiment, the pH can be about 4.

Also, the present formulation does not require a stabilizing antioxidant. A stabilizing antioxidant is a molecule capable of slowing or preventing the oxidation of other molecules. Oxidation is a chemical reaction that transfers electrons from a substance to an oxidizing agent. Oxidation reactions can produce free radicals, which start chain reactions that damage cells. Antioxidants terminate these chain reactions by removing free radical intermediates, and inhibit other oxidation reactions by being oxidized themselves. As a result, antioxidants are often reducing agents such as thiols or polyphenols. More example of antioxidants include but are not limited to a sulfur- and alkali metal-containing antioxidant. Examples of sulfur- and alkali metal-containing antioxidants include but are not limited to sodium metabisulfite, acetone sodium bisulfite and sodium formaldehyde sulfoxylate.

One or more osmotic pressure adjusting agents may be included in the presently disclosed formulation. Osmotic pressure adjusting agents are chemicals which can set the osmotic pressure of a solution. Osmotic pressure is the hydrostatic pressure produced by a solution in a space divided by a semipermeable membrane due to a differential in the concentrations of solute. Inclusion of osmotic pressure adjusting agents may be necessary to match the osmotic pressure of a patient. Examples of such osmotic pressure adjusting agents include but are not limited to mannitol and sodium chloride.

The presently disclosed formulations can be produced as ready-to-use solutions. Whereas, the previously described solutions of elsamitrucin, such as described in U.S. Pat. No. 5,508,628 was lyophilized to obtain the solid form which is reconstituted with a pharmaceutical carrier such as water just prior to administration, the presently disclosed formulations are stable in the liquid form and have a long shelf life as described in the Examples below. Therefore, the presently disclosed solutions may be stored and administered without reconstitution prior to use. The usable formulation of U.S. Pat. No. 5,508,628 requires lyophilization because the in situ formed elsamitrucin salt solution contained residual solvents such as methanol, ethanol, chlorofirm, n-butanol and t-butanol. Freeze-drying (also known as lyophilization or cryodesiccation) is a dehydration process typically used to preserve a perishable material or make the material more convenient for transport. Freeze-drying works by freezing the material and then reducing the surrounding pressure and adding enough heat to allow the frozen water in the material to sublime directly from the solid phase to gas.

Having residual solvents present would be unacceptable for administration into a patient. Lyophilization was necessary to remove these impurities in U.S. Pat. No. 5,508,628. In one embodiment, the presently disclosed formulations do not contain such impurities which are for example: methanol, ethanol, chloroform, n-butanol and t-butanol.

The following Examples are provided as illustrative embodiments of the present invention. It should be understood that the stable dried, or nearly dried elsamitrucin salts of the present invention are not limited by the following examples. The teachings of the Examples that follow can be used by pharmaceutical chemists of ordinary skill as guidance in making other, variations that result in the same compositions as disclosed here.

### EXAMPLES

### Example 1

### Initial Preparation of the Stable Elsamitrucin Salts of the Present Disclosure

Small batches of elsamitrucin salts were prepared prior to optimization and scale up. Eight counter ions based on organic acids were selected, these included lactic acid, maleic acid, succinic acid, L-tartaric acid, p-toluenesulfonic (also referred to herein as p-TSA or tosylate), benzoic acid, salicylic acid, and sulfuric acids. Three solvents were selected based on previous screen methods known to those skilled in the art of pharmaceutical chemistry, the selected solvents included dioxane, dimethylformamide (DMF), and acetic acid (AcOH). An additional combination of p-TSA/MeOH was included for a total of twenty-five variations reactions.

To each reaction vial, 3.0×10⁻⁵ mol of elsamitrucin base was added. The elsamitrucin base was dissolved in 0.25 mL of DMF or AcOH at 55° C., 1.5 mL of dioxane at 80° C., or 12 mL of MeOH at 70° C. and stirred for five minutes to ensure dissolution. Each vial was then charged with 245-270 μL of a 0.126 M dioxane solution of one of the organic acids listed above (see Table 1) corresponding to 1.05 equivalent of each of the eight acids (tartaric acid was dispensed in a 1:1 mixture of methanol/water due to its insolubility in dioxane).

TABLE 1

| | | A- Dioxane (1.5 mL) | | B - DMF (0.25 mL) | | C- AcOH (0.25 mL) | | D - MeOH (12 mL) | |
|---|---|---|---|---|---|---|---|---|---|
| | | API | Acid sol. | API | Acid sol. | API | Acid sol. | API | Acid sol. |
| | Acid | (mg) | 0.126M(mL) | (mg) | 0.126M(mL) | (mg) | 0.126M(mL) | (mg) | 0.126M(mL) |
| 1 | Lactic | 20.25 | 0.260 | 20.01 | 0.255 | 19.13 | 0.245 | — | — |
| 2 | Maleic | 19.96 | 0.255 | 21.22 | 0.270 | 20.23 | 0.260 | — | — |
| 3 | Succinic | 20.42 | 0.260 | 19.18 | 0.245 | 19.12 | 0.245 | — | — |
| 4 | L-Tartaric | 20.65 | 0.265 | 19.55 | 0.250 | 19.62 | 0.250 | — | — |
| 5 | p-TSA | 19.52 | 0.250 | 19.41 | 0.250 | 19.61 | 0.250 | 21.08 | 0.270 |
| 6 | Benzoic | 19.63 | 0.250 | 21.03 | 0.270 | 19.96 | 0.255 | — | — |
| 7 | Salicylic | 19.95 | 0.255 | 21.04 | 0.270 | 20.15 | 0.260 | — | — |
| 8 | Sulfuric | 19.54 | 0.250 | 21.17 | 0.270 | 19.97 | 0.255 | — | — |

API = elsamitrucin base.

The initial temperature was held for ten minutes and then ramped down to room temperature at a rate of 20° C./hour for DMF and AcOH, 30° C./hour for dioxane and 25° C./hour for MeOH. Solids were formed in the vials with dioxane/L-tartaric acid, dioxane/p-TSA, dioxane/sulfuric acid, and AcOH/sulfuric acid. Solids were collected by filtration and dried in vacuo at 50° C. and 30 in. Hg. Vials were solids did not form were concentrated to dryness with a stream of nitrogen and dried in vacuo at 50° C. and 30 in. Hg. Methanol was removed under vacuum and the resultant residue dried under high vacuum at room temperature. All samples were analyzed by x-ray diffraction (XRPD), differential scanning calorimetry (DSC), and thermogravimetric analysis (TGA) to determine the crystallinity of the salts. Crystalline solids were obtained from dioxane/sulfuric acid and from AcOH/sulfuric acid; semi-crystalline solids were obtained from dioxane/L-tartaric acid, dioxane/p-TSA, DMF/lactic acid, DMF/maleic acid, DMF/L-tartaric acid, DMF/benzoic acid, DMF/sulfuric acid, AcOH/lactic acid, AcOH/p-TSA, and AcOH/benzoic acid. All other solids were found to be amorphous by XRPD.

### Example 2

### Optimization of Elsamitrucin Salt Preparation

Next three elsamitrucin salts made in accordance with the teaching of Example 1 were selected for scale-up development. The salts selected were elsamitrucin tartrate, elsamitrucin sulfate and elsamitrucin tosylate. These were selected because each provided crystalline or semi-crystalline solids that precipitated during the cooling process, which can allow better isolation and purification (if necessary) of the salt and thus lends them more suitable for larger scale manufacturing techniques. However, their selection for purposes of Example 2 should not be considered a limitation.

L-tartaric acid, sulfuric acid and p-TSA were dissolved in dioxane. Suitable reaction containers were each charged with 1.7×10⁻⁴ mol of elsamitrucin base which was dissolved in 7.5 mL of dioxane at 80° C., and stirred for five minutes to ensure dissolution. Each vial was then charged with 350-380 μL of a 0.5 M solution of the organic acid in dioxane corresponding to approximately 1.05 equivalent each of the three acids (Table 2).

TABLE 2

| | Elsamitrucin | | Amount | Acid | Amount |
|---|---|---|---|---|---|
| Exp. # | (mg) | Solvent | (mL) | (0.5 M) | (mL) |
| 1 | 118 | Dioxane | 9.0 | Sulfuric | 0.38 |
| 2 | 113 | Dioxane | 7.0 | p-TSA | 0.36 |
| 3 | 108 | Dioxane | 7.5 | L-Tartaric | 0.35 |

The initial temperature was held for ten minutes and then ramped down to room temperature at a rate of 30° C./hour for dioxane. Solids were formed upon addition of acid to the vials with dioxane/L-tartaric acid and dioxane/sulfuric acid and for dioxane/p-TSA precipitation occurred during the cooling process. After filtration the solids were dried in vacuo at 50° C. and 30 inch Hg. Samples were analyzed by XRPD, DSC, and TGA to determine the crystallinity (Table 3), and other physical properties.

As Table 3 shows, all solids in Example 2 were semi-crystalline, contained up to about 5% of the residual solvent and were pasty in constancy due to the high amount of solvent that was retained in the solids due to a rapid precipitation.

The elsamitrucin salts of the present disclosure were also prepared using a slower precipitation method than described above. Reaction containers were charged with 7.6×10⁻⁵ mol of elsamitrucin base and 5 mL of dioxane at 80° C. After the mixture was stirred for five minutes to ensure dissolution of the base, 400 μL of a 0.2 M aqueous solution of tartaric acid corresponding to 1.05 equivalents was added to the dissolved elsamitrucin base. The temperature was held at 80° C. for ten minutes and then the vials were cooled to room temperature at a rate of 30° C./hour. During the cooling phase precipitation occurred. The solids were collected by filtration and dried in vacuo at 50° C. and 30 inches Hg. Samples were analyzed by XRPD, DSC, and TGA to determine physical properties [Table 3, OVL-A-55(1) and OVL-A-55(2)]. The first sample [dioxane/sulfuric acid, OVL-A-55(1)] was crystalline by XRPD, but it contained 3.6% residual solvent according to TGA analysis and three endothermic peaks on the DSC curve. The second sample [dioxane/L-tartaric acid, OVL-A-55(2)] was semi-crystalline.

Next, elsamitrucin salts were prepared in an aqueous environment as follows. Reaction vials were charged with 100 mg of elsamitrucin base, 1.05 equivalents of corresponding acid (p-TSA, succinic, and L-tartaric acid were added as solids; sulfuric acid was dissolved in 0.5 mL of water) and water (10 mL for p-TSA, succinic, and L-tartaric acid, 9.5 mL for sulfuric acid). The suspensions were heated to 80° C. with stirring for ten minutes to form a clear solution and then ramped down to room temperature at a rate of 30° C./hour. After stirring overnight at room temperature precipitates were not formed in any of the experiments. The water was removed under a gentle flow of nitrogen at 35° C. Precipitation was observed in the p-TSA experiment after the removal of one-third of the water, this solid was filtered and dried in vacuo at 50° C. and 30 inches Hg. The filtrate was also analyzed. The other three vials were evaporated to dryness and dried in vacuo at 50° C. and 30 inches Hg. The results showed that the solids produced were semi-crystalline with high amorphous content [Table 3, OVL-A-47(1), OVL-A-47(2-1), OVL-A-47(3), and OVL-A-65].

TABLE 3

| | | | | | | DSC Analysis | | | TGA wt |
|---|---|---|---|---|---|---|---|---|---|
| Lab Notebook | | | Second | Drying | | Ramp | Onset | Peak | loss |
| Lot Number | Salt | Solvent | Solvent | Conditions | XRPD | ° C./min | (° C.) | (° C.) | (%) |
| — | Free base | — | | 25° C. in | Crystalline | 10 | | 52.5 | 5.3 |
| | | | | vacuo | | | | 138.4 | |
| | | | | | | | | 202.2 | |
| | | | | | | | | 210.2 | |
| OVL-A-45(1) | Sulfuric | Dioxane | | 50° C. in | Semicrys | 10 | | 66.7 | 1.3 |
| | | | | vacuo | | | | 139.2 | |
| | | | | | | | | 170.0(x) | |
| | | | | | | | | 191.2 | |
| OVL-A-45(3) | p-TSA | Dioxane | | 50° C. in | Semicrys | 10 | | 63.0 | 5.0 |
| | | | | vacuo | | | 147.7 | 155.8 | |
| | | | | | | | | 175.0(x) | |
| | | | | | | | | 184.0 | |
| OVL-A-45(4) | L-Tartaric | Dioxane | | 50° C. in | Semicrys | 10 | | 90.7 | 5.6 |
| | | | | vacuo | | | 192.9 | 202.3 | |
| | | | | | | | | 198.23 | |
| | | | | | | | 240.6 | 249.83 | |
| OVL-A-47(1) | Sulfuric | Water | | 50° C. in | Semicrys | 10 | | 139.0(x) | |
| | | | | vacuo | | | | 172.0(x) | |
| OVL-A-47(2a) | p-TSA | Water | | 50° C. in | Semicrys | 10 | | 66.0 | |
| | | | | vacuo | | | | 182.0(x) | |
| | | | | | | | | 186.0 | |
| OVL-A-47(2b) | p-TSA | Water | | 50° C. in | Semicrys | 10 | | 139.0(x) | |
| | | | | vacuo | | | | 172.0(x) | |
| OVL-A-47(3) | L-Tartaric | Water | | 50° C. in | Semicrys | 10 | | 91.0 | |
| | | | | vacuo | | | | | |
| | | | | | | | | 184.0 | |
| | | | | | | | | 202.0 | |
| OVL-A-51(1b) | Sulfuric | Dioxane | EtOAc | 50° C. in | Semicrys | 10 | 44.6 | 75.0 | |
| | | | | vacuo | | | 176.5 | 179.0(x) | |
| OVL-A-51(2b) | p-TSA | Dioxane | EtOAc | 50° C. in | Amorphous | 10 | | 184.0 | |
| | | | | vacuo | | | | 186.0(x) | |
| | | | | | | | | 189.0 | |
| OVL-A-51(1c) | Sulfuric | Dioxane | Heptane | 50° C. in | Amorphous | 10 | | 67.0 | |
| | | | | vacuo | | | 164.4 | 173.0 | |
| OVL-A-51(2c) | p-TSA | Dioxane | Heptane | 50° C. in | Amorphous | 10 | | 67.0 | |
| | | | | vacuo | | | | 159.0 | |
| | | | | | | | | 169.0(x) | |
| | | | | | | | | 182.0 | |
| OVL-A-51(3c) | L-Tartaric | Dioxane | Heptane | 50° C. in | Amorphous | 10 | | 63.0 | |
| | | | | vacuo | | | | | |
| | | | | | | | 162.1 | 187.0 | |
| | | | | | | | 255.2 | 266.0 | |
| OVL-A-55(1) | Sulfuric | Dioxane | | 50° C. in | Crystalline | 10 | 58.7 | 78.0 | 3.6 |
| | | | | vacuo | | | 199.7 | 205.0 | |
| | | | | | | | | 209.0 | |
| OVL-A-55(2) | L-Tartaric | Dioxane | | 50° C. in | Semicrys | 10 | | 75.0 | |
| | | | | vacuo | | | 195.9 | 205.0 | |
| | | | | | | | | 209.0 | |
| OVL-65 | Succinic | Water | | 50° C. in | Semicrys | 10 | | 88.0 | |
| | | | | vacuo | | | 158.8 | 173.0 | |
| | | | | | | | | 189.0 | |
| | | | | | | | | 210.0 | |
| OVL-67 | Sulfuric | AcOH | MeCN | 50° C. in | Crystalline | 10 | 261.3 | 278.0 | 0.2 |
| | | | | vacuo | | | | | |

(x)-exotherm

### Example 3

### Crystallization of Elsamitrucin Tosylate Salt Using Microscopy

On a microscope slide, 1-2 mg of amorphous elsamitrucin tosylate was sprinkled with the help of a spatula and a cover-slip was placed. Drops of solvent were placed on the side of the cover-slip so as to allow the solvent to sip under the cover-slip and dissolve the drug. The drug in contact with the solvent was stored at room temperature and examined under microscope with 100× or 400× magnification. The solvents used were isopropyl alcohol, methanol, ethanol, acetonitrile, acetone, propylene glycol, tetrahydrofuran, dichloromethane and 1:1 mixtures of isopropyl alcohol, methanol, ethanol, acetonitrile, acetone with water. Needle or rod-shaped crystals were observed under microscope in solvents—ethanol, methanol, propylene glycol, isopropyl alcohol, acetone, and all the water:solvent mixtures. Microscopic examination of the samples indicated that the elsamitrucin tosylate salt became crystalline in at least one solvent.

### Example 4

### Microcrystallization of MSA, p-TSA and HCl Salts of Elsamitrucin

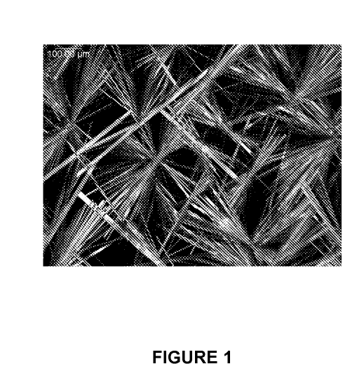

A small amount of the salt (1-2 mg) was placed on a microscope slide and covered with a cover-slip. Several drops of solvent were added at the edge of the cover-slip so that capillary action would draw the solvent between the slide and the cover-slip. If the material was partially dissolved, the slide was slowly heated on a hot-plate until most of the solid had dissolved. Each slide was cooled at room temperature to allow slow crystallization. 1:1 mixtures of methanol, ethanol, isopropyl alcohol and acetonitrile in water were used for the crystallization. Each salt in all the four different solvent systems produced crystals of elsamitrucin. Elsamitrucin tosylate salt crystals showed birefringence under plane polarized light as depicted in FIG. 1.

### Example 5

### Scale Up of Re-Crystallization of p-TSA Salt

The recrystallization of p-TSA salt was carried out with slow evaporation in a Craig tube. The p-TSA salt was dissolved in 1:1 mixture of acetonitrile and water at elevated temperatures. After hot filtration in the Craig tube, the solvent from the reaction was evaporated slowly, which yielded precipitate. Under microscope, the crystal habit was observed to be needle-shaped. The crystals were isolated by filtration and dried under vacuum. The material was observed to be transparent to XRD, which was confirmed to be crystalline by microscopy. In the differential scanning calorimetry analysis, the material showed a melt followed by degradation or crystallization at 183° C. and 186° C., respectively. ¹H NMR analysis showed the sample to be 1:1 ratio of API to the counter ion (p-TSA).

The solubility of the p-TSA salt in water was checked by HPLC after stirring the slurry at room temperature for six hours. It was found that the solubility of the p-TSA salt in water is 15.6 mg/mL (Table 5, lot # OVL-A-137). The solubility of the p-TSA salt at pH 4 (benzoate buffer) is 14.7 mg/mL (Table 5, OVL-A-143).

TABLE 4

| Lab | | Concentration | | Vial | | |
|---|---|---|---|---|---|---|
| Notebook | | of HPLC | Dilution | Concentration | | Concentration |
| Lot Number | Counts | Sample (M) | Factor | (M) | MW Salt | (mg/mL) |
| OVL-A-137 | 1191856 | 9.45269E−05 | 200 | 0.018905385 | 825.83 | 15.612634 |
| OVL-A-143 | 1126074 | 8.9459E−05 | 200 | 0.017891806 | 825.83 | 14.77559 |

Elsamitrucin salts made in accordance with the teachings of the present disclosure were tested for stability. Two samples of the isolated p-TSA salt (40 mg each) were placed in a vacuum oven at 75° C. for nine hours. After this exposure, sample #1 was taken out, the temperature was increased to 98° C. and the second sample was dried overnight. The NMR data showed a perfect 1:1 salt ratio, so there was no decomposition in the solid state during drying at elevated temperatures. The weight loss by TGA was approximately 2.5% for both samples. Karl Fischer analysis indicated the two lots still had water present: sample #1 had 4.0% water content and #2 had 4.6%. The p-TSA salt (16 mg) of elsamitrucin was dissolved in 1.6 mL of benzoate buffer (pH 4) and stirred at 50° C. for ten days. Samples for HPLC and MS were taken in 3, 5, and 10 days. No evidence of the decomposition product was found by either MS (peak at 282) or HPLC.

### Example 6

### Scale-Up of HCl Salt Formation

Elsamitrucin (200 mg) was slurried in 1 mL of acetonitrile/water (1:1) and heated up to 75° C. resulting in a very thick slurry. A 1 M HCl solution in water (0.321 mL, 1.05 eq.) was added to the slurry to form a clear solution. The mixture was then slowly cooled to room temperature at a rate of 25° C./h with very gentle stirring. After stirring at room temperature for approximately 6 hours, the solids obtained were isolated by filtration and dried in vacuo at 50° C. and 30 inches Hg to yield 187.5 mg (88.86% yield) of the HCl salt. DSC and XRD analyses confirmed the crystalline nature of the salt.

### Example 7

### In Vitro Growth Inhibitory Activities of Elsamitrucin and Elsamitrucin Tosylate Salt

The following experiment confirms that the elsamitrucin salts made in accordance with the teachings of the present disclosure retain their in vitro anti-neoplastic activity when compared to elsamitrucin base. Elsamitrucin and elsamitrucin tosylate were tested in vitro using: B16F10 (murine lung), HCT 116 (human colon), HT29 (human colon) and SK-MES-1 (human non-small cell lung carcinoma). Cell growth inhibition was evaluated in 96-well micro-culture plates with a semi-automated MTT (3-(4,5-dimethylthiazol-2-yl)-2,5-diphenyltetrazolium bromide) assay.

SK-MES-1 human non-small cell lung carcinoma, B16F10 murine melanoma cells, HCT 116 and HT29 human colon carcinomas (collectively “test cell cultures”) were maintained in buffered RPMI 1640 supplemented with fetal calf serum, antibiotics and other appropriate growth factors such as glutamine. Test cells (1,500-2,000 cells/well) were seeded in a 96-well micro culture plate with a total volume of 100 μL/well. After overnight incubation in a humidified incubator at 37° C. with 5% CO₂ and 95% air, elsamitrucin solutions were diluted to various concentrations with RPMI 1640, were added to each well in a 100 μL volume. The elsamitrucin base and the elsamitrucin tosylate solutions (elsamitrucin solutions) were prepared and stored in a −20° C. freezer. The solutions were thawed not more than 10 times for the entire experiment.

Cell culture plates seeded with test cells and various concentrations of elsamitrucin solutions were placed in a humidified incubator at 37° C., with CO₂ and 95% air for 5-10 days. The plates were then centrifuged briefly, and 100 μL of the growth medium was removed. The cell cultures were incubated with 50 μL of MU reagent (1 mg/ml in Dulbecco™ phosphate-buffered saline) for 4 hours at 37° C. The resultant purple formazan precipitate was solubilized with 200 μL of 0.04 N HCl in isopropanol. Absorbance was monitored at a wavelength of 595 nm, and at a reference wavelength of 650 nm, using a TECAN® GENios mocroplate reader. In all experiments, absorbance data was acquired for each agent at two overlapping concentration ranges. In most cases, the study was repeated using broader concentration ranges.

The results of each test were stored and imported into PRISM® 3.03 for graphical analysis and determination of IC₅₀ values. All results were graphed as a percentage of controlled absorbance versus the drug concentration. The IC₅₀ values were estimated with PRISM® 3.03 using nonlinear regression analysis to fit the data to the sigmoidal dose-response curve described by the following four-logistic equation:

$$Y = \frac{Top - Bottom}{1 + {( X / {IC}_{50} )}^{n}} + Bottom$$

Top is the maximal percentage of control absorbance, bottom is the minimal percentage of control absorbance at the highest agent concentration, Y is the observed absorbance, X is the agent concentration, IC₅₀ is the concentration of agent that inhibits cell growth by 50% compared to the control cells, and n is the slope of the curve. Table 4 demonstrates that elsamitrucin and elsamitrucin tosylate salt possess essentially the same anti-proliferative effect on the cell lines tested. Thus as demonstrated in Table 4, the elsamitrucin salts made in accordance with the teachings of the present disclosure can be expected to have equivalent, or superior in vivo anti-neoplastic activity as therapeutic compositions made using elsamitrucin base alone. The elsamitrucin tosylate comprises an IC₅₀ that is within preferably about 20%, more preferably about 15% and most preferably about 10% of a similar amount of an elsamitrucin base.

TABLE 5
In Vitro Growth Inhibitory Activities of Elsamitrucin and Elsamitrucin
p-TSA Salt against SK-MES-1 Human Non-small Cell Lung Carcinoma.
B16F10 Murine Melanoma and HCT 116 and HT29 Human Colon
Carcinoma Cells.

| | Elsamitrucin | Elsamitrucin p-TSA Salt |
|---|---|---|
| Cell Line | IC₅₀ (μm) | IC₅₀ (μm) |
| SK-MES-1 | 0.042 | 0.045 |
| B16F10 | 0.024 | 0.028 |
| HCT 116 | 0.074 | 0.079 |
| HT29 | 0.095 | 0.105 |

### Example 8

### Stability of Elsamitrucin Formulation

Elsamitrucin F2 Formulation (10 mg/mL of elsamitrucin free base with 4.77% mannitol at pH 4.0) in a 2.5 mL dosage form was used for the stability study. The formulation was stable in both upright and inverted positions at 5 and 25° C. for a period of 12 weeks and pH for those samples maintained fairly stable in a range of 4.0 to 4.3. However, decrease in pH was observed for those samples stored at elevated temperature as degradation proceeded. Using Arrhenius method, the zero order degradation rate constants (kT) for the upright samples at 5 and 25° C. were roughly estimated as 5.79×10⁻⁵ and 9.84×10⁻⁴ mg/mL per day, respectively. For the inverted samples, the corresponding zero order degradation rate constants were 2.49×10⁻⁵ and 6.28×10⁻⁴ mg/mL per day at 5° C. and 25° C., respectively. This Elsamitrucin F2 Dosage Form was therefore expected to be capable of maintaining their potency greater than 90% for a period of longer than 2.5 years at 25° C. (as compared to 47 years for this dosage form at 5° C. to achieve the same level drop in potency).

The Elsamitrucin F2 RTU Formulation used consists of Elsamitrucin Tosylate: 3.2903 g (equivalent to 10 mg/mL free base in final solution), Mannitol: 11.9251 g, Water For Injection: 250 mL. The pH was set to be 4.0 by using NaOH.

Design of the Stability Study: 250 mL of Elsamitrucin Tosylate stock solution (10 mg/mL free base, 4.77% Mannitol, pH 4.0) was prepared. 80×5 mL amber serum vials were filled with 2.5 mL of the Elsamitrucin Tosylate stock solution, flushed with nitrogen and sealed with stoppers. These sealed vials, in both upright and inverted positions, were stored in the following temperature cabinets at 4, 25, 40 and 60° C., respectively. The toxicity of the stock solution was recorded at time zero. Samples were removed for pH measurement and high performance liquid chromatography (HPLC) analysis at the various time points. At 60° C., measurements were made at 0, 2, 7, 10, 14 days. At 40° C., measurements were made at 0, 7, 14, 28, 38, 64, 77, 84 days. At 25° C., measurements were made at 0, 7, 14, 28, 64, 84 days. At 4° C., measures were made at 0, 7, 14, 28, 64, 84 days.

The apparatus and materials used in the stability study are as follows—Vials: 5 mL Wheaton amber serum vial (Mouth I.D.×O.D.-13×20 mm, part number 223695, Lot #1394689), Stopper: 20-mm Stelmi serum stopper (bromobutyl, gray, part number 6720GC, Lot #B603/18047), Aluminum Seal: 20-mm Wheaton unlined aluminum seal (part #224193-01), Elsamitrucin Tosylate: Albany Molecular Research, Inc., Lot # DKK-M-27, Water For Injection (WFI): Phoenix Pharmaceuticals, Lot #703097F, Mannitol: J. T. Baker, Lot #C39645, 0.2 N Sodium Hydroxide Solution: VWR International, Lot # 7050, 0.22 Micron Cellulose Acetate Membrane Filter Corning Inc., Part #430624, Diffuser: Waters, Part #WAT007272, pH meter: Fisher Scientific Accumet Basic equipped with VWR Symphony Ag/AgCl pH Electrode (calibrated with VWR pH standards at pH=4.01, 7.0 and 10.0), Osmometer: Advanced Instrument Osmometer Model 3320.

Elsamitrucin Tosylate Stock Solution Preparation: 3.2903 g of Elsamitrucin Tosylate and 11.9251 g of mannitol were accurately weighed out in a 250 mL volumetric flask. About 200 mL of Water For Injection, degassed by bubbling nitrogen via a Waters diffuser for 1 hr prior to use, was added to the flask. The mixture was stirred in a water bath at 45-50° C. until all solid dissolved. After cooled to ambient temperature, pH of the solution was adjusted to 4.0 with 0.2 N NaOH. Water For Injection was then added to the mark and the pH of the solution was rechecked. The solution was then filtered through a 0.22 micron cellulose acetate membrane filter and bubbled with nitrogen via a Waters diffuser for 5 minutes. 2.5 mL of the stock solution was transferred to a 5 mL amber serum vial (80×). The headspace for each vial was purged with nitrogen and sealed with the Stemli serum stopper and Wheaton unlined aluminum seal.

For the present stability study HPLC weight/weight assay of elsamitrucin was done in addition to the determination of related impurities. The reagents for the HPLC assay are as follows—HPLC grade acetonitrile and trifluoroacetic acid (Lot #44093418) were obtained from EMD Science. Water was purified with Millipore Milli-Q system. The apparatus was the chromatographic system which consisted of a Waters Alliance 2695 separation module equipped with a column heater, an autosampler and a Waters 2996 photodiode array detector. Data acquisition was controlled by Waters Empower Pro 2 software. As the column a Cadenza Cd-C18, 3 μm, 4.6×150 mm (Silverstone Sciences) was used. Detection was made at 267 nm. Mobile phase A contained water with 0.1% trifluoroacetic acid. 2 mL of trifluoroacetic acid was pipetted to 2000 mL of water. Mobile phase B contained acetonitrile with 0.08% trifluoroacetic acid. 1 mL of trifluoroacetic acid was pipetted to 1250 mL of acetonitrile. 1 L of water was mixed thoroughly with 1 L of acetonitrile.

HPLC analysis was done at various time points as shown below.

TABLE 6
HPLC Analysis Schedule

| Sample | Days/Weeks |
|---|---|
| Stock | 0 day |
| 60° C. | 2 days |
| 4, 25, 40, 60° C. | 7 days |
| 60° C. | 10 days |
| 4, 25, 40, 60° C. | 14 days |
| 4, 25, 40° C. | 28 days |
| 40° C. | 38 days |
| 4, 25, 40, 60° C. | 64 days |
| 40° C. | 77 days |
| 4, 25, 40° C. | 84 days |

The following table shows the gradient conditions:

TABLE 7
Gradient Conditions

| Time (min) | % A | % B | Gradient Mode |
|---|---|---|---|
| 0 | 90 | 10 | Initial |
| 4 | 72 | 28 | Linear |
| 8 | 65 | 35 | Linear |
| 15 | 60 | 40 | Linear |
| 30 | 10 | 90 | Linear |
| 32 | 10 | 90 | Hold |
| 33 | 90 | 10 | Linear |
| 45 | 90 | 10 | Hold |

HPLC analysis was run with a gradient at flow rate equal to 1.0 mL/min. The oven temperature was set at 40° C. The autosampler temperature was at 25° C. The injection volume was 10 μL. Sample Diluent—Acetonitrile/water (50/50, v/v): 1 L of water was mixed thoroughly with 1 L of acetonitrile. Sample Blank—Acetonitrile/water (50/50, v/v): Sample Diluent was used as Sample Blank. Standard Solution—˜0.1 mg/mL of Elsamitrucin: 26.32 mg of Elsamitrucin Tosylate was accurately weighed and dissolved in a 200 mL of Sample Diluent in a 200 mL volumetric flask. Sample Solution Preparation (one vial per time point): 2 mL of Elsamitrucin RTU solution was pipetted to a 20 mL volumetric flask and diluted with 18 mL of Sample Diluent. 2 mL of the diluted solution was further diluted with 18 mL of Sample Diluent in a 20 mL volumetric flask to give the final analytical sample (with concentration at about 0.1 mg/mL). The retention time for Elsamitrucin was about 11.7 minutes.

Immediately after usage, the column was flushed with Solvent B for 30 minutes and followed by Sample Diluent for 45 minutes. The column was stored in Sample Diluent at the end of each use. The mean concentration of Elsamitrucin free base in the stock solution at time zero was found as 10.035 mg/mL with mean osmotic pressure equal to 301.6 mOsm/kg.

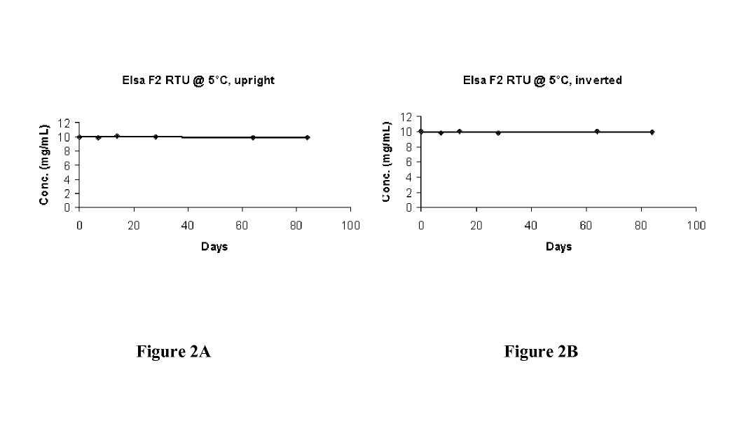

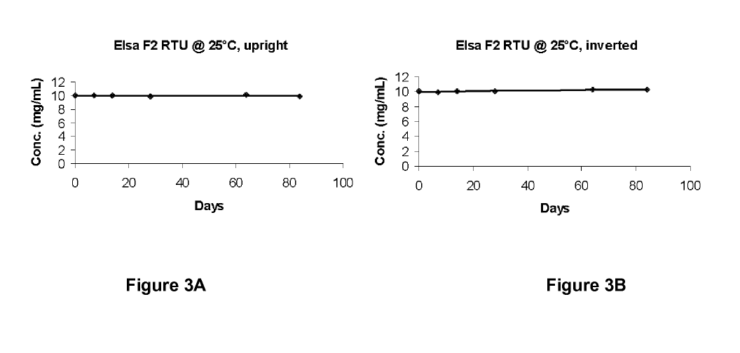

Potency of Elsamitrucin F2 RTU −2.5 mL Dosage Form is show below in: Table 8 which summarize the potency of 2.5 mL Elsamitrucin F2 RTU Dosage Form in upright and inverted positions during a period of 12 weeks. This dosage form was stable at 4 and 25° C. in both storage positions as shown in FIGS. 2A, 2B, 3A and 3B. It showed different degrees of degradation at elevated temperatures. The zero order degradation rate constants (kT) at 313 and 333 K are listed in Table 9.

TABLE 8
Potency of 2.5 mL Elsamitrucin F2 Dosage Form

| | | | | | | Potency of Elsa RTU F2 at | | | |
|---|---|---|---|---|---|---|---|---|---|
| | Potency of Elsa RTU F2 (mg/mL) | | | | | inverted position (mg/mL) | | | |
| Day | 4° C. | 25° C. | 40° C. | 60° C. | Day | 4° C. | 25° C. | 40° C. | 60° C. |
| 0 | 10.035 | 10.035 | 10.035 | 10.035 | 0 | 10.035 | 10.035 | 10.035 | 10.035 |
| 2 | | | | 9.890 | 2 | | | | 9.900 |
| 7 | 9.890 | 9.970 | 9.810 | 9.450 | 7 | 9.860 | 9.960 | 9.930 | 9.570 |
| 10 | | | | 9.230 | 10 | | | | 9.340 |
| 14 | 10.090 | 9.980 | 9.980 | 9.250 | 14 | 10.080 | 9.990 | 10.000 | 9.050 |
| 28 | 10.030 | 9.910 | 9.900 | | 28 | 9.860 | 10.080 | 10.050 | |
| 38 | | | 9.720 | | 38 | | | 9.920 | |
| 64 | 9.880 | 10.120 | 9.440 | | 64 | 10.040 | 10.270 | 9.740 | |
| 77 | | | 9.700 | | 77 | | | 9.700 | |
| 84 | 9.850 | 9.000 | 9.350 | | 84 | 9.900 | 10.290 | 9.510 | |

TABLE 9
The Zero Order Degradation Rate Constants (kT) at 313K and 333K

| | kT (mg/mL per Day) | |
|---|---|---|
| Temperature (K) | upright position | inverted position |
| 313 | 0.0065 | 0.0054 |
| 333 | 0.0618 | 0.0703 |

Determination of Estimated Degradation Rate Constants (k278K & k293K) for 2.5 mL Elsamitrucin F2 RTU Dosage Form: Using the Arrhenius method for the limited data in Table 9, the degradation rate constants for Elsamitrucin F2 RTU Dosage Form in upright position at 5° C. (or 278 K) and 25° C. (or 293 K) were roughly estimated equal to 5.79×10⁻⁶ and 9.84×10⁻⁴ mg/mL per day, respectively. In order to have a 10° A) drop in potency, (i.e. from 10 mg/mL to 9 mg/mL) of Elsamitrucin F2 RTU Dosage Form in upright position, it will take about 17282 days when stored at 5° C. and 1016 days at 25° C.

Similarly, in the inverted position, the estimated degradation rate constants for Elsamitrucin F2 RTU Dosage Form were 2.49×10⁻⁵ and 6.28×10⁻⁴ mg/mL per day at 5° C. and 25° C., respectively. This implies that the potency of Elsamitrucin F2 RTU Dosage Form will decrease 10% when stored in inverted position at 5° C. for a period of 40236 days, while it takes 1593 days when kept at 25° C.

In general, samples of Elsamitrucin F2 RTU Dosage Form stored in inverted position are more stable than those in upright position. Although the reason for the stability discrepancy between two storage positions was not clear, it plausibly relates to the composition of the Stelmi serum stopper which, while in contact of the formulation, does somehow slow down the degradation mechanism(s).

Impurities Profiles: Tables 3-6 list the impurities profile of the 2.5 mL Elsamitrucin F2 RTU Dosage Form. For the vials kept at 5 and 25° C., pH of the formulation was fairly stable (in the range of 4.0 to 4.3) during the testing period. However, drop in pH was observed with the progress of degradation in those samples kept at elevated temperature. The major degradation impurities (derived under a stress condition at 60° C.) were those with the relative retention time as follows: 0.59, 0.62, 1.41, 1.65, 1.82 and 1.84.

TABLE 3
Impurities Profile of Elsamitrucin F2 RTU Dosage Form at 5° C.

| | | 10 mg/mL ELSA RTU - Formulation 2 | | | | | | 10 mg/mL ELSA RTU - Formulation 2 | | | | | |
|---|---|---|---|---|---|---|---|---|---|---|---|---|---|
| | | (in upright position) @ 5° C. | | | | | | (in inverted position) @ 5° C. | | | | | |
| | Relative | Area Percent | | | | | | Area Percent | | | | | |
| | Retention | | Day | Day | Day | Day | Day | | Day | Day | Day | Day | Day |
| Peak | Time | Initial | 7 | 14 | 28 | 64 | 84 | Initial | 7 | 14 | 28 | 64 | 84 |
| Unknown 1 | 0.18 | 0.31 | 0.29 | 0.34 | 0.29 | 0.31 | 0.44 | 0.31 | 0.29 | 0.27 | 0.32 | 0.38 | 0.51 |
| Unknown 2 | 0.20 | 0.48 | 0.37 | 0.39 | 0.40 | 0.59 | 0.49 | 0.48 | 0.45 | 0.43 | 0.14 | 0.45 | 0.17 |
| Unknown 3 | 0.28 | 0.64 | 0.57 | 0.58 | 0.64 | 0.57 | 0.67 | 0.64 | 0.46 | 0.64 | 0.27 | 0.68 | 0.27 |
| Unknown 4 | 0.31 | 0.34 | 0.35 | 0.34 | 0.36 | 0.35 | 0.37 | 0.34 | 0.34 | 0.35 | 0.31 | 0.37 | 0.32 |
| Unknown 5 | 0.45 | | | | 0.08 | | 0.04 | | | | | | |
| Unknown 6 | 0.48 | | | | 0.28 | | | | | | | | |
| Unknown 7 | 0.54 | | | | | | | | | | | | |
| Unknown 8 | 0.56 | | | | | | | | | | | | |
| Unknown 9 | 0.59 | | | | | | | | | | | | |
| Unknown 10 | 0.62 | | | | 0.27 | | | | | | | | |
| Unknown 11 | 0.63 | | | | | | | | | | | | |
| Unknown 12 | 0.84 | | | | | | | | | | | | |
| Unknown 13 | 0.90 | | | | | | | | | | | | |
| Unknown 14 | 0.95 | 0.48 | 0.48 | 0.50 | 0.48 | 0.53 | 0.49 | 0.48 | 0.47 | 0.48 | 0.48 | 0.50 | 0.50 |
| Unknown 15 | 0.97 | | | | | | | | | | | | |
| ELSA | 1.00 | 97.63 | 97.86 | 97.63 | 97.63 | 97.39 | 97.33 | 97.63 | 97.89 | 97.70 | 98.41 | 97.54 | 98.15 |
| Unknown 16 | 1.11 | 0.03 | | 0.04 | 0.04 | 0.04 | 0.03 | 0.03 | | 0.03 | | | |
| Unknown 17 | 1.13 | | | | | | | | | | | | |
| Unknown 18 | 1.19 | | | | | | | | | | | | |
| Unknown 19 | 1.41 | 0.10 | 0.08 | 0.17 | 0.17 | 0.22 | 0.14 | 0.10 | 0.10 | 0.11 | 0.07 | 0.07 | 0.08 |
| Unknown 20 | 1.65 | | | | | | | | | | | | |
| Unknown 21 | 1.78 | | | | | | | | | | | | |
| Unknown 22 | 1.81 | | | | | | | | | | | | |
| Unknown 23 | 1.82 | | | | | | | | | | | | |
| Unknown 24 | 1.84 | | | | | | | | | | | | |
| Unknown 25 | 1.92 | | | | | | | | | | | | |
| Unknown 26 | 2.08 | | | | | | | | | | | | |
| Unknown 27 | 2.13 | | | | | | | | | | | | |
| Unknown 28 | 2.23 | | | | | | | | | | | | |
| | Total | 2.38 | 2.14 | 2.36 | 2.99 | 2.61 | 2.67 | 2.38 | 2.11 | 2.31 | 1.59 | 2.45 | 1.85 |
| | Impurity % | | | | | | | | | | | | |
| | pH | 4.00 | 4.17 | 4.18 | 4.18 | 4.18 | 4.22 | 4.00 | 4.17 | 4.18 | 4.20 | 4.28 | 4.26 |

TABLE 4
Impurities Profile of Elsamitrucin F2 RTU Dosage Form at 25° C.

| | | 10 mg/mL ELSA RTU - Formulation 12 | | | | | | 10 mg/mL ELSA RTU - Formulation 2 | | | | | |
|---|---|---|---|---|---|---|---|---|---|---|---|---|---|
| | | (in upright position) @ 25° C. | | | | | | (in inverted position) @ 25° C. | | | | | |
| | Relative | Area Percent | | | | | | Area Percent | | | | | |
| | Retention | | Day | Day | Day | Day | Day | | Day | Day | Day | Day | Day |
| Peak | Time | Initial | 7 | 14 | 28 | 64 | 84 | Initial | 7 | 14 | 28 | 64 | 84 |
| Unknown 1 | 0.18 | 0.31 | 0.28 | 0.28 | 0.30 | 0.31 | 0.48 | 0.31 | 0.27 | 0.30 | 0.33 | 0.40 | 0.46 |
| Unknown 2 | 0.20 | 0.48 | 0.39 | 0.44 | 0.16 | 0.43 | 0.17 | 0.48 | 0.41 | 0.50 | 0.15 | 0.40 | 0.26 |
| Unknown 3 | 0.28 | 0.64 | 0.51 | 0.68 | 0.27 | 0.62 | 0.26 | 0.64 | 0.52 | 0.59 | 0.27 | 0.62 | 0.91 |
| Unknown 4 | 0.31 | 0.34 | 0.33 | 0.35 | 0.32 | 0.35 | 0.33 | 0.34 | 0.36 | 0.32 | 0.31 | 0.35 | 0.35 |
| Unknown 5 | 0.45 | | | | | | | | | | | | |
| Unknown 6 | 0.48 | | | | | | | | | | | | |
| Unknown 7 | 0.54 | | | | | | | | | | | | |
| Unknown 8 | 0.56 | | | | | | | | | | | | |
| Unknown 9 | 0.59 | | | | | | | | | | | | 0.04 |
| Unknown 10 | 0.62 | | | | | 0.05 | 0.03 | | | | | 0.03 | 0.09 |
| Unknown 11 | 0.63 | | | | | | | | | | | | |
| Unknown 12 | 0.84 | | | | | | | | | | | | |
| Unknown 13 | 0.90 | | | | | | | | | | | | |
| Unknown 14 | 0.95 | 0.48 | 0.50 | 0.49 | 0.49 | 0.50 | 0.51 | 0.48 | 0.51 | 0.49 | 0.49 | 0.50 | 0.48 |
| Unknown 15 | 0.97 | | | | | | | | | | | | |
| ELSA | 1.00 | 97.63 | 97.86 | 97.67 | 98.32 | 97.68 | 98.08 | 97.63 | 97.71 | 97.73 | 98.37 | 97.42 | 97.28 |
| Unknown 16 | 1.11 | 0.03 | 0.03 | | 0.03 | | 0.04 | 0.03 | 0.04 | | | 0.04 | 0.04 |
| Unknown 17 | 1.13 | | | | | | | | | | | | |
| Unknown 18 | 1.19 | | | | | | | | | | | | |
| Unknown 19 | 1.41 | 0.10 | 0.09 | 0.08 | 0.10 | 0.07 | 0.09 | 0.10 | 0.18 | 0.08 | 0.08 | 0.24 | 0.10 |
| Unknown 20 | 1.65 | | | | | | | | | | | | |
| Unknown 21 | 1.78 | | | | | | | | | | | | |
| Unknown 22 | 1.81 | | | | | | | | | | | | |
| Unknown 23 | 1.82 | | | | | | | | | | | | |
| Unknown 24 | 1.84 | | | | | | | | | | | | |
| Unknown 25 | 1.92 | | | | | | | | | | | | |
| Unknown 26 | 2.08 | | | | | | | | | | | | |
| Unknown 27 | 2.13 | | | | | | | | | | | | |
| Unknown 28 | 2.23 | | | | | | | | | | | | |
| | Total | 2.38 | 2.13 | 2.32 | 1.67 | 2.33 | 1.91 | 2.38 | 2.29 | 2.28 | 1.63 | 2.58 | 2.73 |
| | Impurity | | | | | | | | | | | | |
| | % | | | | | | | | | | | | |
| | pH | 4.00 | 4.13 | 4.15 | 4.21 | 4.19 | 4.20 | 4.00 | 4.18 | 4.18 | 4.30 | 4.38 | 4.35 |

TABLE 5
Impurities Profile of Elsamitrucin F2 RTU Dosage Form at 40° C.

| | | 10 mg/mL ELSA RTU - Formulation 2 | | | | | | | | 10 mg/mL ELSA RTU - Formulation 2 | | | | | | | |
|---|---|---|---|---|---|---|---|---|---|---|---|---|---|---|---|---|---|
| | | (in upright position) @ 40° C. | | | | | | | | (in inverted position) @ 40° C. | | | | | | | |
| | Relative | Area Percent | | | | | | | | Area Percent | | | | | | | |
| | Retention | | Day | Day | Day | Day | Day | Day | Day | | Day | Day | Day | Day | Day | Day | Day |
| Peak | Time | Initial | 7 | 14 | 28 | 38 | 64 | 77 | 84 | Initial | 7 | 14 | 28 | 38 | 64 | 77 | 84 |
| Unknown 1 | 0.18 | 0.31 | 0.28 | 0.29 | 0.39 | 0.33 | 0.36 | 0.32 | 0.48 | 0.31 | 0.27 | 0.29 | 0.28 | 0.38 | 0.33 | 0.33 | 0.51 |
| Unknown 2 | 0.20 | 0.48 | 0.38 | 0.50 | 0.17 | 0.16 | 0.56 | 0.58 | 0.16 | 0.48 | 0.45 | 0.51 | 0.16 | 0.15 | 0.52 | 0.45 | 0.31 |
| Unknown 3 | 0.28 | 0.64 | 0.64 | 0.60 | 0.27 | 0.30 | 0.64 | 0.51 | 0.29 | 0.64 | 0.55 | 0.61 | 0.28 | 0.30 | 0.65 | 0.65 | 0.90 |
| Unknown 4 | 0.31 | 0.34 | 0.37 | 0.04 | 0.30 | 0.29 | 0.37 | 0.36 | 0.35 | 0.34 | 0.35 | 0.34 | 0.31 | 0.28 | 0.36 | 0.36 | 0.35 |
| Unknown 5 | 0.45 | | | | 0.03 | 0.10 | 0.08 | 0.03 | 0.06 | | | | 0.04 | 0.04 | 0.08 | 0.07 | |
| Unknown 6 | 0.48 | | | | | | | | | | | | | | | | 0.11 |
| Unknown 7 | 0.54 | | | | | | | | | | | | | | | | |
| Unknown 8 | 0.56 | | | | 0.04 | 0.05 | 0.07 | 0.07 | 0.10 | | | | 0.16 | 0.04 | 0.06 | 0.06 | 0.08 |
| Unknown 9 | 0.59 | | | | 0.07 | 0.06 | 0.06 | 0.06 | 0.08 | | | 0.04 | 0.07 | 0.06 | 0.08 | 0.06 | 0.07 |
| Unknown 10 | 0.62 | | | 0.03 | 0.05 | | | 0.05 | | | 0.06 | | 0.20 | | 0.05 | 0.15 | 0.05 |
| Unknown 11 | 0.63 | | | | | | | | 0.03 | | | | | | | | |
| Unknown 12 | 0.84 | | | | | | | | | | | | | | | | |
| Unknown 13 | 0.90 | | | | | | | 0.17 | 0.07 | | | | | | | 0.13 | 0.04 |
| Unknown 14 | 0.95 | 0.48 | 0.49 | 0.48 | 0.50 | 0.51 | 0.51 | 0.50 | 0.51 | 0.48 | 0.49 | 0.49 | 0.50 | 0.49 | 0.33 | 0.51 | 0.49 |
| Uhknown 15 | 0.97 | | | | | | | | | | | | | | | | |
| ELSA | 1.00 | 97.63 | 97.77 | 97.71 | 97.62 | 97.76 | 96.86 | 96.80 | 97.31 | 97.63 | 97.75 | 97.64 | 97.70 | 98.04 | 97.04 | 96.84 | 96.64 |
| Unknown 16 | 1.11 | 0.03 | | 0.18 | 0.03 | | | | 0.04 | 0.03 | | | 0.03 | 0.03 | 0.04 | 0.03 | |
| Unknown 17 | 1.13 | | | | | | | | | | | | | | | | |
| Unknown 18 | 1.19 | | | | | | | | | | | | | | | | |
| Unknown 19 | 1.41 | 0.10 | 0.08 | 0.10 | 0.30 | 0.07 | 0.06 | 0.33 | 0.09 | 0.10 | 0.08 | 0.09 | 0.08 | 0.07 | 0.07 | 0.11 | 0.06 |
| Unknown 20 | 1.65 | | | 0.03 | 0.06 | 0.08 | 0.12 | 0.10 | 0.15 | | | | 0.05 | 0.05 | 0.14 | 0.12 | 0.17 |
| Unknown 21 | 1.78 | | | | | | | | | | | | | | | | |
| Unknown 22 | 1.81 | | | 0.04 | | 0.04 | 0.04 | 0.03 | 0.04 | | | | | 0.05 | 0.04 | 0.03 | 0.04 |
| Unknown 23 | 1.82 | | | | | | | | | | | | | | | | |
| Unknown 24 | 1.84 | | | | 0.06 | | 0.08 | 0.06 | 0.09 | | | | 0.05 | | 0.09 | 0.07 | 0.10 |
| Unknown 25 | 1.92 | | | | | | | | 0.08 | | | | | | 0.07 | 0.04 | 0.03 |
| Unknown 26 | 2.08 | | | | 0.07 | 0.07 | 0.09 | 0.03 | 0.04 | | | | 0.04 | | 0.06 | | 0.06 |
| Unknown 27 | 2.13 | | | | 0.05 | 0.19 | 0.10 | | 0.03 | | | | 0.05 | | | | |
| Unknown 28 | 2.23 | | | | | | | | | | | | | | | | |
| | Total | 2.38 | 2.24 | 2.29 | 2.39 | 2.25 | 3.14 | 3.20 | 2.69 | 2.38 | 2.25 | 2.37 | 2.30 | 1.94 | 2.97 | 3.17 | 3.37 |
| | Impurity | | | | | | | | | | | | | | | | |
| | % | | | | | | | | | | | | | | | | |
| | pH | 4.00 | 4.13 | 4.15 | 4.22 | 4.18 | 4.00 | 3.88 | 3.85 | 4.00 | 4.14 | 4.18 | 4.28 | 4.28 | 4.17 | 4.16 | 4.10 |

TABLE 6
Impurities Profile of Elsamitrucin F2 RTU Dosage Form at 60° C.

| | | 10 mg/mL ELSA RTU - Formulation 12 | | | | | 10 mg/mL ELSA RTU - Formulation 2 | | | | |
|---|---|---|---|---|---|---|---|---|---|---|---|
| | | (in upright position) @ 60° C. | | | | | (in inverted position) @ 60° C. | | | | |
| | Relative | Area Percent | | | | | Area Percent | | | | |
| | Retention | | Day | Day | Day | Day | | Day | Day | Day | Day |
| Peak | Time | Initial | 2 | 7 | 10 | 14 | Initial | 2 | 7 | 10 | 14 |
| Unknown 1 | 0.18 | 0.31 | 0.27 | 0.31 | 0.33 | 0.36 | 0.31 | 0.27 | 0.32 | 0.35 | 0.36 |
| Unknown 2 | 0.20 | 0.48 | 0.35 | 0.43 | 0.46 | 0.47 | 0.48 | 0.37 | 0.42 | 0.39 | 0.45 |
| Unknown 3 | 0.28 | 0.64 | 0.55 | 0.61 | 0.55 | 0.61 | 0.64 | 0.58 | 0.61 | 0.66 | 0.63 |
| Unknown 4 | 0.31 | 0.34 | 0.37 | 0.37 | 0.37 | 0.38 | 0.34 | 0.35 | 0.37 | 0.37 | 0.38 |
| Unknown 5 | 0.45 | | | 0.11 | 0.10 | 0.05 | | | 0.04 | 0.04 | 0.06 |
| Unknown 6 | 0.48 | | | | | 0.09 | | | | | |
| Unknown 7 | 0.54 | | | 0.05 | 0.08 | 0.10 | | | | 0.03 | 0.04 |
| Unknown 8 | 0.56 | | | 0.10 | 0.15 | 0.13 | | 0.03 | 0.08 | 0.09 | 0.14 |
| Unknown 9 | 0.59 | | 0.05 | 0.13 | 0.17 | 0.15 | | 0.05 | 0.10 | 0.11 | 0.14 |
| Unknown 10 | 0.62 | | | 0.08 | 0.06 | 0.07 | | 0.03 | 0.05 | 0.04 | 0.09 |
| Unknown 11 | 0.63 | | | | 0.08 | 0.06 | | | | | 0.04 |
| Unknown 12 | 0.84 | | | | 0.09 | 0.07 | | | | 0.08 | 0.03 |
| Unknown 13 | 0.90 | | | | 0.15 | 0.08 | | | | 0.04 | 0.80 |
| Unknown 14 | 0.95 | 0.48 | 0.48 | 0.48 | 0.50 | 0.51 | 0.48 | 0.49 | 0.49 | 0.49 | 0.50 |
| Unknown 15 | 0.97 | | | | | 0.07 | | | | | 0.07 |
| ELSA | 1.00 | 97.63 | 97.83 | 96.93 | 95.99 | 95.79 | 97.63 | 97.73 | 96.86 | 96.82 | 96.03 |
| Unknown 16 | 1.11 | 0.03 | | | 0.04 | 0.04 | 0.03 | | 0.18 | | 0.04 |
| Unknown 17 | 1.13 | | | | | 0.09 | | | | | |
| Unknown 18 | 1.19 | | | | 0.22 | 0.20 | | | | | |
| Unknown 19 | 1.41 | 0.10 | 0.07 | 0.07 | 0.07 | 0.07 | 0.10 | 0.09 | 0.08 | 0.06 | 0.16 |
| Unknown 20 | 1.65 | | | 0.10 | 0.14 | 0.16 | | | 0.11 | 0.07 | 0.12 |
| Unknown 21 | 1.78 | | | | 0.05 | 0.05 | | | | | 0.04 |
| Unknown 22 | 1.81 | | | 0.04 | 0.07 | 0.05 | | | 0.10 | 0.06 | 0.07 |
| Unknown 23 | 1.82 | | | | 0.08 | 0.11 | | | | 0.08 | 0.15 |
| Unknown 24 | 1.84 | | | 0.05 | 0.09 | 0.11 | | | 0.05 | 0.07 | 0.19 |
| Unknown 25 | 1.92 | | | | 0.10 | | | | | 0.09 | 0.10 |
| Unknown 26 | 2.08 | | | 0.07 | 0.07 | 0.11 | | | 0.10 | 0.05 | 0.09 |
| Unknown 27 | 2.13 | | 0.03 | 0.03 | | | | | | | |
| Unknown 28 | 2.23 | | | 0.04 | | | | | 0.06 | | |
| | Total | 2.38 | 2.17 | 3.07 | 4.02 | 4.19 | 2.38 | 2.26 | 3.16 | 3.17 | 3.97 |
| | Impurity % | | | | | | | | | | |
| | pH | 4.00 | 4.04 | 3.74 | 3.64 | 3.55 | 4.00 | 4.06 | 3.91 | 3.64 | 3.60 |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements The terms “a” and “an” and “the” and similar references used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of any and all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A formulation comprising a solution of:
at least one stable solid elsamitrucin salt, and
a pharmaceutically acceptable carrier.

2. The formulation of claim 1, wherein said formulation does not contain a buffer to maintain a solution pH.

3. The formulation of claim 1, wherein said formulation does not require a stabilizing antioxidant.

4. The formulation of claim 1, further comprising an osmotic pressure adjusting agent.

5. The formulation of claim 4, wherein said osmotic pressure adjusting agent is mannitol.

6. The formulation of claim 1, further comprising an agent to set the pH between about 3.5 to 4.5.

7. The formulation of claim 1, further comprising an agent to set the pH between about 3.0 to about 4.0.

8. The formulation of claim 1, further comprising an agent to set the pH at about 4.0.

9. The formulation of claim 1, wherein said stable solid elsamitrucin salt is selected from the group consisting of elsamitrucin lactate, elsamitrucin fumarate, elsamitrucin maleate, elsamitrucin succinate, elsamitrucin tartrate, elsamitrucin tosylate, elsamitrucin methanesulfonate, elsamitrucin benzoate, elsamitrucin salicylate, elsamitrucin hydrochloride, elsamitrucin sulfate, and elsamitrucin phosphate.

10. The formulation of claim 9, wherein said elsamitrucin salt is elsamitrucin tosylate.

11. The formulation of claim 1, wherein said pharmaceutically acceptable carrier is water or saline.
